(19) United States
(12) Patent Application Publication
(10) Pub. No.: US 2025/0275773 A1
(43) Pub. Date: Sep. 4, 2025

(54) OFFSET INSTRUMENTATION FOR GLENOID REAMING

(71) Applicants: Smith & Nephew, Inc., Memphis, TN (US); Smith & Nephew Orthopaedics AG, Zug (CH)

(72) Inventors: ARMODIOS M. HATZIDAKIS, Denver, CO (US); DREW MILLER, Atlanta, GA (US); JAMES D. KELLY, II, SAN FRANCISCO, CA (US); SCOTT R. JACOBSON, Bend, OR (US); HEINZ R. HOENECKE, JR., Del Mar, CA (US); Brian Bowman, Carlsbad, CA (US); John Crosby, Cordova, TN (US)

(21) Appl. No.: 18/593,218

(22) Filed: Mar. 1, 2024

(51) Int. Cl. A61B 17/17; A61B 90/00

(52) U.S. Cl. CPC A61B 17/17; A61B 17/1778; A61B 90/03; A61B 2090/036

(57) ABSTRACT

Systems and methods for reaming bone can include an elongate body having a proximal end, a distal end, and an inner throughbore extending therebetween. An angled guide can be configured to engage the distal end of the elongate body and define a bearing surface at a predetermined angle relative to a centerline of the elongate body. A drive shaft can be configured to extend through the inner throughbore of the elongate body, the drive shaft having a distal end configured to extend beyond the distal end of the elongate body. A reamer head can be pivotably coupled to the distal end of the drive shaft, the reamer head having a distal-facing cutting surface configured to cut bone and a proximal-facing portion configured to bear against the bearing surface of the angled guide to orient the reamer head at the predetermined angle.

### FIELD OF THE DISCLOSURE

The present disclosure relates generally to orthopedic devices and methods and more particularly to an instrumentation set arranged and configured to set an angle for version-correcting reaming during, for example, a glenoid reaming procedure.

### BACKGROUND OF THE DISCLOSURE

Many surgical procedures require preparation of a bone surface to receive an implant. In the case of a shoulder procedure, such as a total shoulder replacement, a humeral prosthesis is used to replace the natural head of the patient's humerus. The humeral prosthesis typically includes an elongated post component that is implanted into the intramedullary canal of the patient's humerus and a hemispherically-shaped prosthetic head component that is secured to the post component. In such a total shoulder replacement procedure, the natural glenoid surface of the scapula is typically resurfaced or otherwise replaced with a glenoid component that provides a bearing surface upon which the prosthetic head component of the humeral prosthesis articulates.

In cases in which the patient's natural shoulder, including its soft tissue, has degenerated to a severe degree of joint instability and pain, however, it can be necessary to change the mechanics of the shoulder. In such cases, a reverse shoulder implant configuration can be used in which the prosthetic head, often referred to as a glenosphere component, is secured to the patient's scapula, and the corresponding concave bearing, often referred to as a humeral cup, is secured to the patient's humerus.

In either configuration, it is critical to have the back side surface of the glenoid implant mate closely with a prepared surface on the glenoid bone to prevent implant loosening. When the shoulder is in use, it creates a rocking motion on glenoid implants which can be made worse if there is a gap caused by the surfaces not mating well. To achieve this mating surface, a glenoid bone can be prepared to receive a shoulder implant using a power-driven bone reamer. Various shoulder implants may have different geometries, however, based on any of a variety of factors, including but not limited to the anatomy of the patient, the shoulder being treated (e.g., left shoulder vs. right shoulder), the anatomical bone preparation location (e.g., superior vs. posterior), the intended future use of the implant by the patient, the handedness of the surgeon (e.g., left-handed vs. right-handed), and/or other preferences of the surgeon. Further, multiple types of implants may be able to be used with the same patient, and the determination as to which implant is best may not be made until after the surgical procedure begins, such as after the surgeon has assessed the bone surfaces internally as part of the procedure. The type, size, and shape of the implant can impact how the bone surface must be prepared.

Current reamers are not easily adaptable, however, for different anatomies or bone preparation changes that may arise. For example, existing glenoid reamers typically operate over a guidewire and are only able to use a cutting head having a single axis of rotation that is not easily adjustable to a different axis of rotation. Further, because it may be desirable to use multiple cutting head configurations and/or one or more single cutting heads that have multiple configurations (e.g., different axes of rotation that can be used) with a single base tool, it would be desirable for glenoid reamers to be more adaptable and allow for more interchangeability than they currently do. Providing for a single base tool can allow some components to be disposed of after a single use; other components can be sterilized and reused, or any combination of all disposable or all reusable components. Existing reamers are often complicated and/or difficult to assemble and disassemble while still providing for consistent use.

It would be beneficial to provide an instrumentation set and corresponding method of use that enables surgeons to offset the reamer and driver construct around the glenoid anatomy based on particular needs of the patient and surgery. It is with respect to these and other considerations that the present disclosure may be useful. In addition, an easy to assemble and disassemble instrumentation set can ensure procedures are as efficient as possible.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In some examples, an instrument for reaming bone can include an elongate body having a proximal end, a distal end, and an inner throughbore extending therebetween. An angled guide can be configured to slide axially onto the distal end of the elongate body, the angled guide defining a bearing surface at a predetermined fixed angle relative to a centerline of the elongate body. A drive shaft can be configured to extend through the inner throughbore of the elongate body. In some examples, the drive shaft has a proximal end configured to couple to a power source and a distal end configured to extend beyond the distal end of the elongate body. A reamer head can be pivotably coupled to the distal end of the drive shaft, the reamer head having a distal-facing cutting surface configured to cut bone and a proximal-facing portion configured to bear against the bearing surface of the angled guide to orient the reamer head at the predetermined angle.

In some further examples, a method for reaming bone can include arranging a drive shaft through an inner throughbore of an elongate body, the elongate body having a proximal end and a distal end, the drive shaft being pivotably coupled to a reamer head at a distal end of the drive shaft. The method can further include arranging one of a plurality of angled guides between the distal end of the elongate body and the reamer head. In some examples, each of the plurality of angled guides defines a bearing surface at a different predetermined angle relative to a centerline of the elongate body. The method can then include coupling a proximal end of the drive shaft to a power source and operating the power source to rotate the drive shaft. In this way, the reamer head can bear against the bearing surface of the one of the plurality of angled guides such that the reamer head rotates at the predetermined angle.

In any preceding or subsequent examples, the distal end of the elongate body can include a protrusion extending radially from the distal end, and the angled guide can include a keyway configured to engage the protrusion to retain the angled guide at a fixed rotational position relative to the elongate body.

In any preceding or subsequent examples, the angled guide can include a body portion having a first end configured to engage the distal end of the elongate body, a second end comprising the bearing surface, and a channel formed between the first end and the second end configured to receive the drive shaft therethrough. In some examples, a slot is arranged in the body portion parallel to the bearing surface, the slot being configured to retain the reamer head at the predetermined angle.

In any preceding or subsequent examples, the angled guide can be selected from a plurality of angled guides that each define a different predetermined angle. In some examples, the reamer had a full-wedge reamer configuration, and the plurality of angled guides each define a predetermined angle within a range of about 0° to about 20°. In other examples, the reamer has a half-wedge reamer configuration, and the plurality of angled guides each define a predetermined angle within a range of about 0° to about 40°.

In any preceding or subsequent examples, the drive shaft can be cannulated from the proximal end to the distal end. In such examples, the drive shaft can be configured to slide over a pin driven into a center of a bone surface to be reamed.

In any preceding or subsequent examples, the reamer head can include a shoulder stop configured to prevent the reamer head from reaming more than a predetermined depth.

In any preceding or subsequent examples, a locking collar can be configured to releasably secure the drive shaft at a position within the inner throughbore of the elongate body corresponding to the reamer head being arranged at a desired position beyond the distal end of the elongate body.

In any preceding or subsequent examples, one or more of the elongate body, the angled guide, the drive shaft, or the reamer head are composed of a material that is sterilizable between multiple reaming operations.

Examples of the present disclosure provide numerous advantages. For example, the instrumentation set according to the present disclosure is easily adaptable for different anatomies or bone preparation changes that may arise. The disclosed examples also have fewer components than conventional instrument configurations, which can lead to faster assembly and easier cleaning. In addition, the overall design may aid in ensuring less bone removal than prior art.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 illustrates side perspective views of an instrumentation set in various stages of assembly in accordance with one or more features of the present disclosure.

FIG. 2 illustrates a side cutaway view of an elongate body of an instrumentation set in accordance with one or more features of the present disclosure.

FIG. 3 illustrates a side cutaway view of a drive shaft and reamer head of an instrumentation set in accordance with one or more features of the present disclosure.

FIG. 4A illustrates a side cutaway view of a reamer head of an instrumentation set in accordance with one or more features of the present disclosure.

FIG. 4B illustrates a side view of a reamer head of an instrumentation set in accordance with one or more features of the present disclosure.

FIGS. 5A-5D illustrate various views of an angled guide of an instrumentation set in accordance with one or more features of the present disclosure.

FIG. 6 illustrates a side view of an instrumentation set including a half-wedge reamer head and arm mechanism in accordance with one or more features of the present disclosure.

FIG. 7 is a side perspective view of an arm mechanism of an instrumentation set in accordance with one or more features of the present disclosure.

FIG. 8 is a side cutaway view of an angled guide of an instrumentation set in accordance with one or more features of the present disclosure.

FIG. 9 is a side perspective view of an instrumentation set in use for reaming a bone in accordance with one or more features of the present disclosure.

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

Various features or the like of an instrumentation set will now be described more fully herein with reference to the accompanying drawings, in which one or more features of the instrumentation set will be shown and described. It should be appreciated that the various features may be used independently of, or in combination, with each other. It will be appreciated that the instrumentation set as disclosed herein may be embodied in many different forms and may selectively include one or more concepts, features, or functions described herein. As such, the instrumentation set should not be construed as being limited to the specific examples set forth herein. Rather, these examples are provided so that this disclosure will convey certain features to those skilled in the art.

In accordance with one or more features of the present disclosure, an instrumentation set including a reamer head is disclosed in which the axis of rotation of the reamer head is adjustable. The angle of the head is adjustable via a fixed angle component with which the reamer engages. In some examples, the fixed angle is set such that it ensures the bone is removed at a mating angle to the implant. The mating angle can be selected by choosing from a set of fixed angle components that each define one of a set of predetermined angles to approximately match patients, or the angle can be set based on the selection of a fixed angle component that is designed to define a custom angle corresponding to the patient anatomy for a custom fit ream and custom fit implant. Although the present disclosure will describe the instrumentation set in accordance with performing glenoid reaming, it is envisioned that the instrumentation set may be used in other applications.

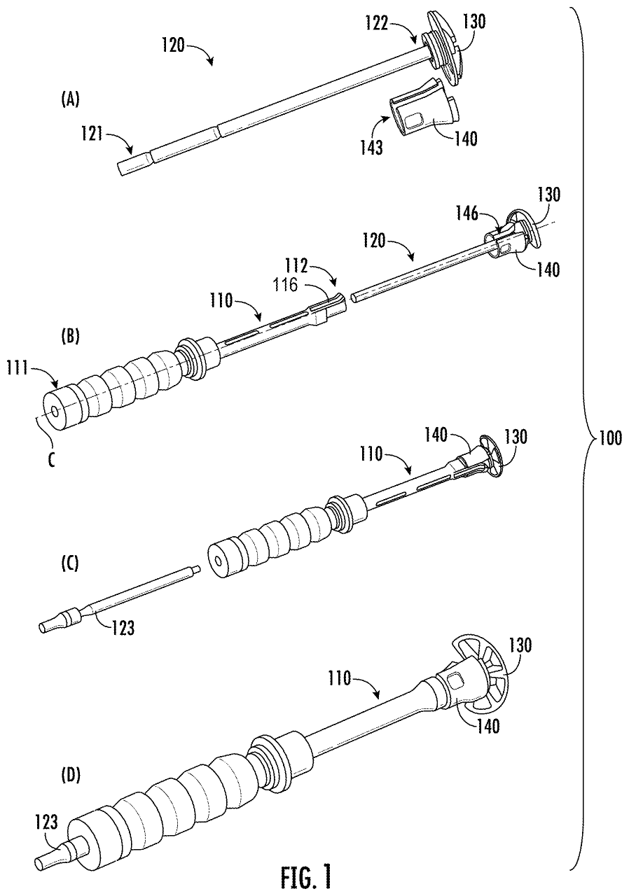

In accordance with one or more features of the present disclosure, with reference to FIG. 1, the instrumentation set 100 includes an elongate body, generally designated 110. In some examples, the elongate body 110 can include a proximal end 111, a distal end 112, and an inner throughbore 113 extending therebetween. The instrument set 100 can further include a drive shaft 120 configured to extend through the inner throughbore 113 of the elongate body 110. In some examples, the drive shaft 120 includes a proximal end 121 that is rotatably supported within the throughbore 113 and is configured to couple to a power source. The proximal end 111 can connect to a driver element 123 inserted into the proximal end 111 of the elongate body 110, and the driver element 123 can be configured to be driven by a power drill. The drive shaft 120 can further include a distal end 122 configured to extend beyond the distal end 112 of the elongate body 110. This distal end 112 can be configured to receive a reamer head. The instrument set 100 further includes a reamer head 130 that is coupled to the distal end 122 of the drive shaft 120 at a pivot point such that the reamer head 130 can both spin about the pivot point and pitch and/or roll to any of a range of angular positions with respect to the distal end 122. In some examples, the reamer head 130 has a proximal-facing portion 131 that is coupled to the drive shaft 120 and a distal-facing cutting surface 132 opposing the proximal-facing portion 131 that is configured to cut bone.

The reamer head 130 can be arranged and held at an angle relative to a centerline C of the instrument set 100 using an angled guide 140. In some examples, the angled guide 140 is configured to engage the distal end 112 of the elongate body 110 and define one or more bearing surface 142 at a predetermined angle a relative to a neutral cutting plane N orthogonal to the centerline C of the instrument set 100. For example, if an x degree angled guide is used, the bearing surface 142 can be arranged at that angle such that the cutting surface 132 of the reamer head 130 is tilted by x degrees with respect to the neutral cutting plane N that is perpendicular to the longitudinal or the centerline C. Stated otherwise, the x degree angled guide arranges the reamer head 130 such that it rotates about an offset axis of rotation O that is tilted by x degrees relative to the centerline C.

When assembled, the proximal-facing surface 131 of the reamer head 130 can be configured to bear against the one or more bearing surface 142 of the angled guide 140 to orient the reamer head 130 at the predetermined angle. In this regard, the angled guide 140 does not support the drive shaft 120 and reamer head 130 with respect to the elongate body 110 but rather adjusts the position of the reamer head 130. In some examples, the angled guide 140 can be selected from a plurality of angled guides that each define different predetermined angles. Exemplary angles may range from about 0° to about 20° for full-wedge reamer configurations and between about 0° and about 40° for half-wedge reamer configurations. The plurality of angled guides can be provided as a standard set that includes different angled guides defining angles corresponding to common reaming angles that are able to approximately match patients, or other configurations for the angle guide 140 can be provided at an angle that is specifically designed based on the patient anatomy for a custom fit ream and custom fit implant. In this way, the ability to quickly adapt the instrument set 100 to use differently angled cutting head positions enables surgeons to select the offset/angle that the reamer and driver construct around the glenoid anatomy based on the particular needs of the patient and surgery.

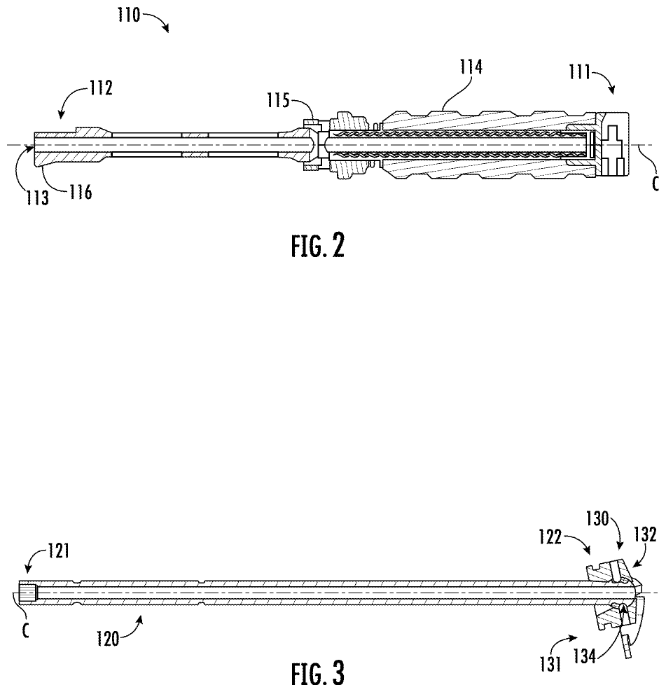

Each of the elements of the instrument set 100 can further include features configured to improve the ability of the reamer head 130 to prepare the bone surface to receive an implant. Referring to FIG. 2, the elongate body 110 can include a handle 114 provided at or near the proximal end 111 that is configured to be gripped by the operator. In use, the elongate body 110 can be manufactured from any suitable rigid material such as, for example, any surgical metal such as, for example, titanium, titanium alloys, stainless steel, cobalt-chromium alloys, tantalum, or the like.

The elongate body 110 can be configured to receive the drive shaft 120 into the inner throughbore 113 at the distal end 112 of the elongate body 110. In some examples, the elongate body 110 can include a locking collar 115 or similar retention mechanism that is configured to releasably secure the drive shaft 120 to the distal end 112 of the elongate body 110 at a position within the inner throughbore 113 corresponding to the reamer head 130 being arranged at a desired position beyond the distal end 112 of the elongate body 110. In this way, the locking collar 115 can be moved to disengage a locking element that at least partially obstructs the inner throughbore 113, the drive shaft 120 can be inserted into the inner throughbore 113 (e.g., through the distal end 112 of the elongate body 110), and the locking collar 115 can further be moved to engage the locking element to secure the drive shaft 120 to the distal end 112 of the elongate body 110. When engaged, the locking collar 115 can be configured to permit a small amount of axial movement of the drive shaft 120, but the locking collar 115 can be configured to retain and rotatably support the drive shaft 120 within the inner throughbore 113 unless and until the locking collar 115 is operated to disengage the locking element to release the drive shaft 120.

In some examples, the locking collar 115 can be provided in the form of a spring-loaded, quick-connect-type fitting. Such a quick-connect feature can allow for easy disassembly to change among the plurality of angled guides 140 and/or to clean components after surgery. For example, the surgeon may pull back to locking collar 115 (e.g., against the biasing force of a spring) and insert the drive shaft 120 into the distal end 112 of the elongate body 110 until it is fully seated within the inner throughbore 113. In some examples, the surgeon may move the locking collar 115 back to an engaged position to engage the locking element or the biasing element may apply a force to return the locking collar 115 to the engaged position when the drive shaft 120 is fully seated within the inner throughbore 113. In either configuration, the locking collar 115 can serve to retain the drive shaft 120 within the throughbore 113.

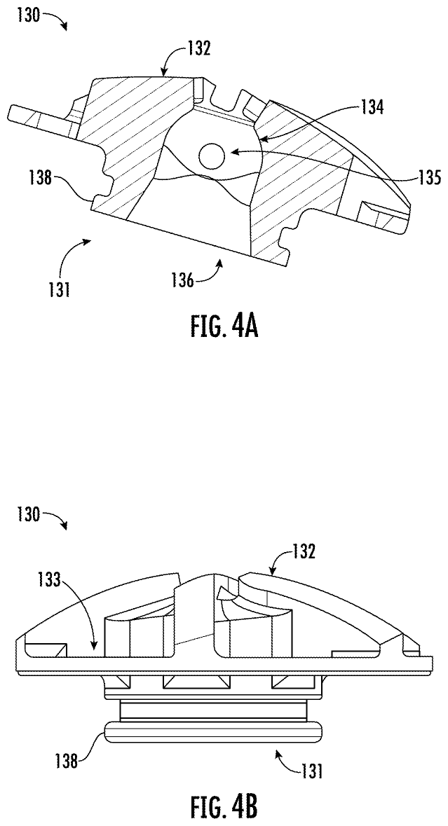

Referring to FIGS. 3-4B, the reamer head 130 can be selected to have a diameter that is sized to remove just enough bone material for the profile of the implant to prevent damage to the soft tissue. In some examples, the shape of the reamer head 130 is a full-wedge design that defines a spherical segment to more closely match the shape of the glenoid. In addition, in some examples, the reamer head 130 may include a shoulder stop 133, such as is shown in FIG. 4B, which can be configured to help to prevent the reamer head 130 from reaming beyond a predetermined depth. This configuration ensures that more cortical (i.e., hard) bone is left behind for maximum stability.

As discussed above, the reamer head 130 can be configured to be arranged with respect to the drive shaft 120 so that it is operable at the offset axis of rotation O that is tilted relative to centerline C of the drive shaft 120. In some examples, this arrangement can be advantageous where the drive shaft 120 is cannulated to slide over a guidewire/pin 150 driven into the location to be reamed, such as is shown in FIG. 9. Referring to FIG. 4A, in some examples, the reamer head 130 is coupled to the drive shaft 120 by a form of universal joint 134 that translates rotation of the drive shaft 120 about a centerline C of the instrument set into rotation about any of a range of angles relative to the centerline C. To accommodate this selective pivoting relative to the drive shaft 120, the universal joint 134 defines a pivot point 135 within the body of the reamer head 130 that couples the reamer head 130 at the distal end 112 of the drive shaft 120 but allows the reamer head 130 to pitch and/or roll to any of a range of angles with respect to the centerline C of the drive shaft 120. In some examples, the proximal-facing portion 131 includes a substantially frustoconical opening 136 into which the distal end 122 of the drive shaft 120 can be inserted to couple to the reamer head 130 at the pivot point 135 at any of a range of angles.

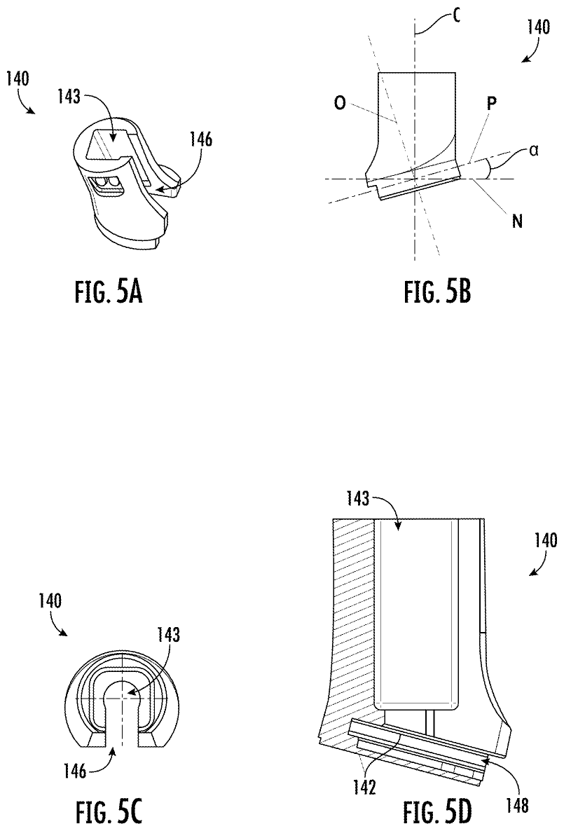

As discussed above, the reamer head 130 can be held at a desired angle a relative to a centerline C of the instrument set 100 using an angled guide 140. Referring to FIGS. 5A and 5C, in some examples, the angled guide 140 defines a channel 143 that extends through the body of the angled guide 140 and is configured to surround the drive shaft 120. In some examples, the channel 143 is sized to be larger than a diameter of the drive shaft 120 so that the drive shaft 120 can freely rotate within the channel 143 with respect to the angled guide 140. By arranging the angled guide 140 about the drive shaft 120, the instrument set 100 can allow the operator to select the desired angle of offset in a more compact form than conventional offset reamers, which typically require a secondary holding mechanism that is parallel to the drive shaft but offset directionally so that changing the offset angle is achieved by correspondingly changing the length of the secondary member. In some examples, the angled guide 140 includes an opening 146 that extends along the length of the angled guide. In some examples, the opening 146 is wider than a diameter of the drive shaft 120 such that the drive shaft 120 can be inserted through the opening 146 until the drive shaft 120 is aligned within the channel 143. Alternatively, in other examples, the opening 146 is narrower than the diameter of the drive shaft such that the drive shaft 120 is arranged in the channel 143 of the angled guide 140 by inserting the proximal end 121 of the drive shaft 120 into the channel 143 and sliding the angled guide 140 toward the distal end 122 of the drive shaft 120.

In some examples, the angled guide 140 can define the bearing surface 142 at a predetermined angle relative to the centerline C. As shown in FIGS. 5B and 5D, the predetermined angle can be characterized by an augment angle « between the bearing surface 142 and a plane orthogonal to the centerline C. In some examples, the plurality of angled guides can be provided with this augment angle « at a few selected angles that can generally be useful for a variety of patients, such as 0°, 5°, 10°, or 15°. Alternatively, the angled guide 140 can be selected to have a specific augment angle a based on the patient anatomy for a custom fit ream and custom fit implant. In some examples, the augment angle a of a given angled guide 140 can be fixed at any of a range of values, including but not limited to angles between about 0° and about 20° for full-wedge reamer configurations and between about 0° and 40° for half-wedge reamer configurations.

To securely position the reamer head 130 at the desired angle, the angled guide 140 can further be configured to retain the reamer head 130 in the desired orientation when the instrument set 100 is fully assembled. With further reference to FIGS. 5B and 5D, in some examples, such as is shown in FIG. 5D, the angled guide 140 includes a slot 148 that substantially surrounds the channel 143 that is likewise accessed through the opening 146. Referring to FIGS. 4A and 4B, the reamer head 130 can correspondingly include a ridge or other surface feature 138 at the proximal-facing portion 131 that is sized and configured to slide within the slot 148. In this arrangement, assembly of the angled guide 140 with the drive shaft 120 can include inserting the ridge 138 into the slot 148 prior to or concurrently with the drive shaft 120 being inserted into the channel 143. In some examples, the slot 148 can include one or more surface feature that is configured to provide tactile and/or auditory feedback during insertion of the ridge 138 until the ridge 138 is fully seated within the slot 148, thereby providing a “snap-on” effect. The drive shaft 120 and the reamer head 130 are thus free to rotate with respect to the angled guide 140, but the reamer head 130 is maintained at a fixed angle with respect to the centerline C. In addition, as discussed above, in some examples, the drive shaft 120 is cannulated from the proximal end 121 to the distal end 122 such that the drive shaft 120 (and reamer head 130) can slide over a guidewire/pin 150 that is inserted at the location to be reamed. In this way, the desired orientation of the reamer head 130 with respect to the bone surface to be reamed can maintained throughout the procedure.

To further secure the angled guide 140 between the elongate body 110 and the reamer head 130 at the desired orientation, the elongate body 110 can include a protrusion 116 extending radially from the distal end (See, e.g., FIG. 2), and the opening 146 formed in the angled guide 140 can function as a corresponding keyway that is configured to engage the protrusion 116 to retain the angled guide 140 at a fixed rotational position relative to the elongate body 110. In some examples, the angled guide 140 is configured to slide axially onto the distal end 112 of the elongate body 110. To engage the angled guide 140 with the elongate body 110, the opening 146 can be aligned with the protrusion 116 during this axial coupling such that the protrusion 116 nests within the opening 146. In this arrangement, the elongate body 110 is designed to lock the reamer head 130 in place but allow rotation while protecting the surgeons hand from the spinning component.

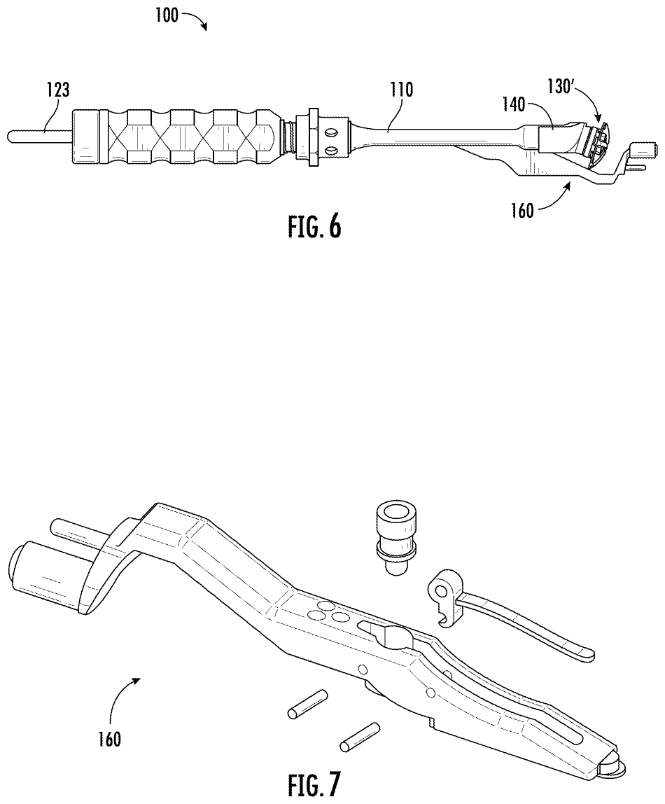

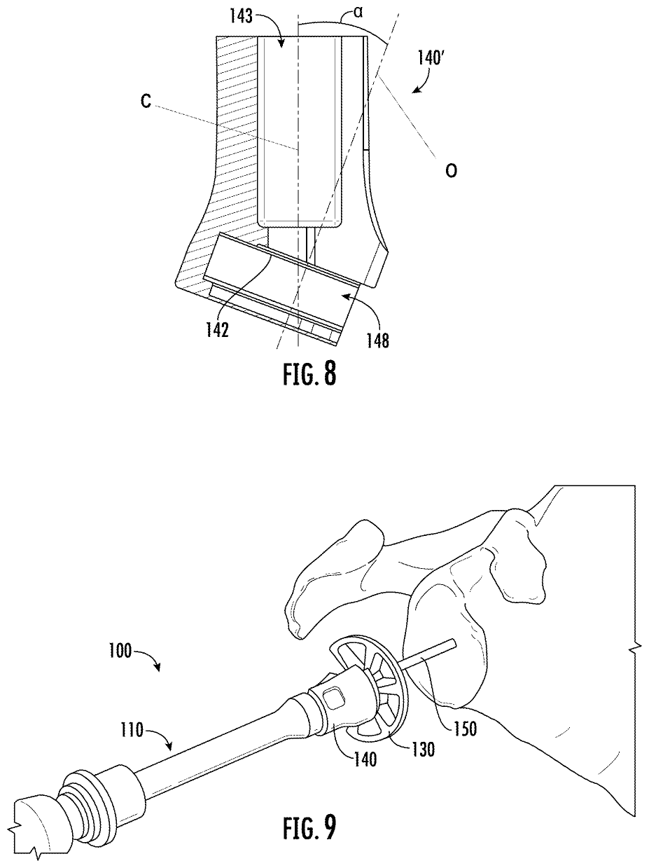

In an alternative configuration shown in FIGS. 6-8, the instrumentation set 100 can be configured for reaming using a half-wedge reamer head 130′. In some examples, an angled guide 140 having a bearing surface 142 arranged at an augment angle a of 0 degrees can first be used to create a primary cut. The half-wedge reamer head 130′ can then be used in subsequent steps to create a secondary spherical segment at a desired angle. In such configurations, a modified angled guide 140′ can be used to arrange the half-wedge reamer head 130′ at the desired offset angle a. In such an example, the slot 148 configured to receive the ridge 138 formed at the proximal end 131 of the reamer head 130′ can be wider than in the full-wedge configuration to accommodate the different head design, which can be arranged in a wider range of angles. The benefit of such a configuration is that even less bone is removed than the full wedge reamer described above.

Similar to the configuration discussed above, a half-wedge reamer head 130′ can be connected to the drive shaft 120. In addition, however, in some examples, the drive shaft 120 can be kept centered with an arm mechanism 160 that engages with the hole that is drilled for the central peg of the anatomic poly glenoid or the reverse baseplate post. In some examples, the arm mechanism 160 is used as a guide to set the reaming depth. The angle of the half-wedge reamer head 130′ is adjustable via an angled guide 140′ with which the half-wedge reamer head 130′ engages. The augment angle a is set such that it ensures the bone is removed at a mating angle to the implant. The half-wedge reamer head 130′ can include a shoulder stop 133 that ensures that more cortical bone is left behind for maximum stability. The shape of the half-wedge reamer head 130′ can again be a spherical segment that substantially matches the shape of the glenoid, and the diameter of the half-wedge reamer head 130′ is sized to remove just enough for the profile of the implant to prevent damage to the soft tissue.

In either configuration, in accordance with one or more features of the present disclosure, a method for reaming bone using such an instrumentation set 100 is further provided. The method can include selecting one of the plurality of angled guides 140 and arranging the angled guide 140 between the distal end 112 of the elongate body 110 and the reamer head 130. As discussed above, in some examples, the selected angled guide 140 can first be slid onto the distal end 122 of the drive shaft 120 such that the drive shaft 120 is arranged in the channel 143 of the angled guide and the ridge 138 at the proximal end 132 of the reamer head 130 is arranged in the slot 148 formed in the angled guide 140.

The method can then further include arranging the drive shaft 120 through the inner throughbore 113 of the elongate body 110. In some examples, this insertion can include aligning the protrusion 116 formed at the distal end 112 of the elongate body 110 with the opening 146 formed in the angled guide 140. In this way, the angle defined by the bearing surface 142 of the angled guide 140 can be held in a fixed orientation with respect to the elongate body 110. The insertion of the drive shaft 120 into the inner throughbore 113 of the elongate body 110 can further include manipulating the locking collar 115 to allow the drive shaft 120 to be fully inserted into the throughbore 113 and securing the drive shaft 120 in the fully inserted position with the locking collar 115.

The method can further include coupling the proximal end 121 of the drive shaft 120 to a power source and operating the power source to rotate the drive shaft 120. In this way, the reamer head 130 bears against the bearing surface 142 of the angled guide 140 such that the reamer 130 head rotates at the desired angle.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

Moreover, the following claims are hereby incorporated into this Detailed Description by this reference, with each claim standing on its own as a separate example of the present disclosure.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one,” “one or more,” and “and/or,” as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

What is claimed is:

1. An instrument for reaming bone, comprising:
an elongate body having a proximal end, a distal end, and an inner throughbore extending therebetween;
an angled guide configured to slide axially onto the distal end of the elongate body, the angled guide defining a bearing surface at a predetermined fixed angle relative to a centerline of the elongate body;
a drive shaft configured to extend through the inner throughbore of the elongate body, the drive shaft having a proximal end configured to couple to a power source and a distal end configured to extend beyond the distal end of the elongate body; and
a reamer head pivotably coupled to the distal end of the drive shaft, the reamer head having a distal-facing cutting surface configured to cut bone and a proximal-facing portion configured to bear against the bearing surface of the angled guide to orient the reamer head at the predetermined angle.

2. The instrument of claim 1, wherein the distal end of the elongate body comprises a protrusion extending radially from the distal end; and
wherein the angled guide comprises a keyway configured to engage the protrusion to retain the angled guide at a fixed rotational position relative to the elongate body.

3. The instrument of claim 1, wherein the angled guide comprises a body portion having a first end configured to engage the distal end of the elongate body, a second end comprising the bearing surface, and a channel formed between the first end and the second end configured to receive the drive shaft therethrough; and
wherein a slot is arranged in the body portion parallel to the bearing surface, the slot being configured to retain the reamer head at the predetermined angle.

4. The instrument of claim 1, wherein the angled guide is selected from a plurality of angled guides that each define a different predetermined angle.

5. The instrument of claim 4, wherein the reamer head comprises a full-wedge reamer configuration, and the plurality of angled guides each define a predetermined angle within a range of about 0° to about 20°.

6. The instrument of claim 4, wherein the reamer head comprises a half-wedge reamer configuration, and the plurality of angled guides each define a predetermined angle within a range of about 0° to about 40°.

7. The instrument of claim 1, wherein the drive shaft is cannulated from the proximal end to the distal end.

8. The instrument of claim 1, wherein the reamer head comprises a shoulder stop configured to prevent the reamer head from reaming more than a predetermined depth.

9. The instrument of claim 1, comprising a locking collar configured to releasably secure the drive shaft at a position within the inner throughbore of the elongate body corresponding to the reamer head being arranged at a desired position beyond the distal end of the elongate body.

10. The instrument of claim 1, wherein one or more of the elongate body, the angled guide, the drive shaft, or the reamer head are composed of a material that is sterilizable between multiple reaming operations.

11. An instrument kit for reaming bone, comprising:
an elongate body having a proximal end, a distal end, and an inner throughbore extending therebetween, wherein the distal end of the elongate body comprises a protrusion extending radially from the distal end;
a plurality of angled guides that are each configured to engage the distal end of the elongate body, wherein each of the plurality of angled guides comprises a keyway configured to engage the protrusion to retain a selected one of the plurality of angled guides at a fixed rotational position relative to the elongate body, and wherein each of the plurality of angled guides defines a bearing surface at a different predetermined angle relative to a centerline of the elongate body;
a drive shaft configured to extend through the inner throughbore of the elongate body, the drive shaft having a proximal end configured to couple to a power source and a distal end configured to extend beyond the distal end of the elongate body, wherein the drive shaft is cannulated from the proximal end to the distal end; and
a reamer head pivotably coupled to the distal end of the drive shaft, the reamer head having a distal-facing cutting surface configured to cut bone and a proximal-facing portion configured to bear against the bearing surface of the angled guide to orient the reamer head at the predetermined angle.

12. The instrument kit of claim 11, wherein the reamer head comprises a shoulder stop configured to prevent the reamer head from reaming more than a predetermined depth.

13. The instrument kit of claim 11, comprising a locking collar configured to releasably secure the drive shaft and the reamer head to the distal end of the elongate body.

14. The instrument kit of claim 11, wherein the reamer head comprises a full-wedge reamer configuration, and each different predetermined angle is within a range of about 0° to about 20°.

15. The instrument kit of claim 11, wherein the reamer head comprises a half-wedge reamer configuration, and each different predetermined angle is within a range of about 0° to about 40°.

16. A method for reaming bone, the method comprising:
arranging a drive shaft through an inner throughbore of an elongate body, the elongate body having a proximal end and a distal end, the drive shaft being pivotably coupled to a reamer head at a distal end of the drive shaft;
arranging one of a plurality of angled guides between the distal end of the elongate body and the reamer head, each of the plurality of angled guides defining a bearing surface at a different predetermined angle relative to a centerline of the elongate body;
coupling a proximal end of the drive shaft to a power source; and
operating the power source to rotate the drive shaft, wherein the reamer head bears against the bearing surface of the one of the plurality of angled guides such that the reamer head rotates at the predetermined angle.

17. The method of claim 16, wherein arranging the drive shaft through the inner throughbore of an elongate body comprises:
disengaging a locking collar arranged on the elongate body to open the inner throughbore;
inserting the drive shaft into the inner throughbore at the distal end of the elongate body; and
engaging the locking collar to secure the drive shaft to the distal end of the elongate body.

18. The method of claim 16, wherein arranging the one of the plurality of angled guides between the distal end of the elongate body and the reamer head comprises aligning a keyway formed in the one of the plurality of angled guides with a protrusion extending radially from the distal end.

19. The method of claim 16, wherein arranging the one of the plurality of angled guides between the distal end of the elongate body and the reamer head comprises positioning the drive shaft in a channel formed in the one of the plurality of angled guides; and
engaging a proximal-facing portion of the reamer head in a slot arranged in the one of the plurality of angled guides at the predetermined angle.

20. The method of claim 16, wherein the drive shaft is cannulated along its length; and
wherein the method comprises sliding the drive shaft over a pin driven into a center of a bone surface to be reamed.
